(19) United States
(12) Patent Application Publication
(10) Pub. No.: US 2006/0287325 A1
(43) Pub. Date: Dec. 21, 2006

(54) Combinations of paroxetine and 2-(r)-(4-fluoro-2-methyl-phenyl)-4-(s)-((8as)-6-oxo-hexahydro-pyrrolo'1,2-a!-pyrazin-2-yl)-piperidine-1-carboxylicacid'1-(r)-(3,5-bis-trifluoromethyl-phenyl)-

(76) Inventor: Sergio Melotto, Verona (IT)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/552,982

(22) PCT Filed: Apr. 16, 2004
(86) PCT No.: PCT/EP04/04121

§ 371 Date: Jun. 12, 2006

(30) Foreign Application Priority Data
Apr. 17, 2003 (GB) 0308968.7

(51) Int. Cl. A61K 31/498; A61K 31/137

(52) U.S. Cl. 514/249; 514/649

(57) ABSTRACT

The present invention relates to therapeutic combinations comprising paroxetine or physiologically acceptable salts or solvates thereof and 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof, to pharmaceutical compositions containing said combinations and their use in the treatment of depression and/or anxiety.

The present invention relates to therapeutic combinations comprising paroxetine or physiologically acceptable salts or solvates thereof and 2-(R-(4Fluoro-2-methyl-phenyl)-4-(S)((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof, to pharmaceutical compositions containing said combinations and their use in the treatment of depression and/or anxiety.

Paroxetine ((−) trans-4-(4′-fluorophenyl)3-(3′-4′-methylenedioxyphenoxymethyl)piperidine) and its salts are commercially available and approved for use in humans for treatment and prophylaxis of, inter alia, anxiety, depression, obsessive compulsive disorder (OCD), premenstrual dysphroic disorder(PMDD) and panic disorders.

2-(R-(4Fluoro-2-methyl-phenyl)-4-(S)-((8aS)-6-oxo-hexahydro-pyrrolo[1,2-a]-pyrazin-2-yl)-piperidine-1-carboxylic acid [1-(R-(3,5bis-trifluoromethyl-phenyl)ethyl]-methylamide or pharmaceutically acceptable salts or solvates thereof, which is described in WO 03/066635, is a NK₁ receptor antagonist.

NK₁ receptor antagonists are known to be useful in the treatment of anxiety and depression, chemotherapy-induced nausea and vomiting and post-operative nausea and vomiting. Preclinical data suggest that NK₁ receptor antagonists may be useful in a variety of other disorders including pain, inflammatory diseases, allergic disorders, CNS disorders, skin disorders, cough and gastrointestinal disorders.

U.S. Pat. No. 6,117,855 describes the use of a CNS-penetrant NK, receptor antagonist together with antidepressant or anti-anxiety drug for the manufacture of a medicament for the treatment or prevention of depression and/or anxiety.

There is however no specific disclosure of such combinations with paroxetine.

WO 03/066635 broadly teaches that the NK₁ receptor antagonists described therein may be administered in combination with a SSRI agent However, there is no teaching concerning any synergistic effect of such combinations in the treatment of depression and/or anxiety.

It has now been found that, surprisingly, therapeutic compositions comprising a combination of paroxetine or physiologically acceptable salts or solvates thereof, for administration in combination with 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)ethyl]-methylamide or pharmaceutically acceptable salts or solvates thereof to a human for the treatment of depression and/or anxiety, in which the dosage of the individual components are administered below the usual single therapeutic dosages, show surprising synergistic levels of efficacy for the treatment and/or prophylaxis of depression and/or anxiety.

In particular, it has now been found that by combining a therapeutically non-effective dose of paroxetine or physiologically acceptable salts or solvates thereof, and a therapeutically non-effective dose of 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide or pharmaceutically acceptable salts or solvates thereof a significantly greater antidepressant activity and/or anxiolytic activity than either of the two individual components taken alone is achieved.

It is a feature of this invention that the use of such a combination will provide one or more of the following effects: a more efficacious anti-depressive and/or anti-anxiety drug and/or a better tolerated drug treatment and/or a drug with a more rapid onset of the anti-depressive and/or anti-anxiety activity.

Furthermore, the synergistic effect of the combination of the present invention allows better management of any potential drug-related side effects.

According to one aspect of the invention, there is provided a combination comprising a therapeutically non-effective dose of paroxetine or physiologically acceptable salts or solvates thereof, and a therapeutically non-effective dose of 2-(R)-(4-Fluoro-2-methyl-phenyl)-4-(S-((8aS)-6-oxo-hexahydro-pyrrolo[1,2-a]-pyrazin-2-yl)piperidine-1-carboxylic acid [-1-(R)-(3,5-bis-trifluoromethyl-phenyl)-ethyl]-methylamide or pharmaceutically acceptable salts or solvates thereof.

When used in any of the contexts or aspects of the present invention a therapeutically non-effective dose refers to a dosage of each component of the combination which is lower than normally expected to produce effective therapeutic response when each component is administered alone.

When used in any of the contexts or aspects of the present invention, paroxetine or physiologically acceptable salts or solvates thereof, may be administered as the free base, or in the form of any physiologically acceptable salt thereof, including all hydrated or anhydrous forms and all polymorphic forms of such salts. In particular, references to paroxetine or physiologically acceptable salts or solvates thereof, include, without limitation, paroxetine hydrochloride, paroxetine hydrochloride hemihydrate, paroxetine hydrochloride anhydrate, paroxetine mesylate and all polymorphic forms thereof.

Paroxetine is preferably used in the form of its hydrochloride hemihydrate salt.

Suitable pharmaceutically acceptable salts of 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide include acid addition salts formed with pharmaceutically acceptable organic or inorganic acids, for example hydrochlorides, hydrobromides, sulphates, alkyl- or arylsulphonates (e.g. methanesulphonates or p-toluenesulphonates), phosphates, acetates, citrates, succinates, tartrates, fumarates and maleates.

Preferred physiologically acceptable salts of 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide include hydrochloride, methanesulphonate, sulphate, p-toluensulphonate.

2-(R)-(4-Fluoro-2-methyl-phenyl)-4-(S)((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide is preferably used in the form of its hydrochloride salt.

According to the invention a therapeutically non-effective dose of 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof, may be in the range of 0.25 to 1 mg per day (measured as the free base) preferably in the range of 0.5 to 1 mg per day.

According to the invention a therapeutically non-effective dose of paroxetine or physiologically acceptable salts or solvates thereof ( measured as the free base) may be in the range of 1 to 10 mg per day, preferably in the range of 3.5 to 7.5 mg per day.

A combination according to the invention conveniently comprises paroxetine or physiologically acceptable salts or solvates thereof, (measured as the free base) in an amount from 1 mg to 10 mg, more particularly in an amount from 3.5 mg to 7.5 mg, and 2-(R)-(4-Fluoro-2-methyl-phenyl) (S-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof, in an amount from 0.25 mg to 1 mg (measured as the free base) and particularly in an amount from 0.5 mg to 1 mg

A preferred combination according to the invention comprises paroxetine or physiologically acceptable salts or solvates thereof, in an amount from 1 to 10 mg (measured as the free base) and 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide or pharmaceutically acceptable salts or solvates thereof, in an amount from 0.25 to 1 mg (measured as the free base).

A preferred combination according to the invention comprises paroxetine or physiologically acceptable salts or solvates thereof, in an amount from 1 to 10 mg (measured as the free base) and 2-(R)-(4Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof, in an amount from 0.5 to 1 mg (measured as the free base).

A preferred combination according to the Invention comprises paroxetine or physiologically acceptable salts or solvates thereof, in an amount from 3.5 to 7.5 mg (measured as the free base) and 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof, in an amount from 0.25 to 1 mg (measured as the free base).

A preferred combination according to the invention comprises paroxetine or physiologically acceptable salts or solvates thereof, in an amount from 3.5 to 7.5 mg (measured as the free base) and 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide or pharmaceutically acceptable salts or solvates thereof, in an amount from 0.5 to 1 mg (measured as the free base).

The dose employed according to the present invention will of course depend on the method of administration, the age, the weight and condition of the patient.

The present invention thus provides a method for the treatment of depression and/or anxiety in a mammal including a human, which comprises treating said animal with a therapeutically effective amount of a combination comprising a therapeutically non-effective dose of paroxetine or physiologically acceptable salts or solvates thereof, and a therapeutically non-effective dose of 2-(R)-(4-Fluoro-2-methyl-phenyl)-4-(S)((8aS)-6-oxo-hexahydro-pyrrolo[1,2-a]-pyrazin-2-yl)-piperidine-1-carboxylic acid [1-(R)(3,5-bis-trifluoromethyl-phenyl)ethyl]-methylamide or pharmaceutically acceptable salts or solvates thereof.

In a further preferred aspect, the present invention provides a method for the treatment of depression and/or anxiety in a mammal including a human, which comprises treating said mammal with a therapeutically effective amount of a combination comprising a therapeutically non-effective dose of paroxetine and a therapeutically non-effective dose of 2-(R)-(4-Fluoro-2-methyl-phenyl)-4-(S((8aS)6-oxo-hexahydro-pyrrolo[1,2-a]-pyrazin-2-yl)piperidine-1-carboxylic acid [1-(R)-(3,5-bis-trifluoromethyl-phenyl)-ethyl]-methylamide hydrochloride.

In a further preferred aspect, the present invention provides a method for the treatment of depression and/or anxiety in a mammal including a human, which comprises treating said mammal with a therapeutically effective amount of a combination comprising a therapeutically non-effective dose of paroxetine hydrochloride and a therapeutically non-effective dose of 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide hydrochloride.

In a further preferred aspect, the present invention provides a method for the treatment of depression and/or anxiety in a mammal including a human, which comprises treating said mammal with a therapeutically effective amount of a combination comprising a therapeutically non-effective dose of paroxetine hydrochloride hemihydrate and a therapeutically non-effective dose of 2-(R)-(4-Fluoro-2-methyl-phenyl)4-(S)-((8aS)-6-oxo-hexahydro-pyrrolo[1,2-a]-pyrazin-2-yl)-piperidine-1-carboxylic acid [1-(R)(3,5bis-trifluoromethyl-phenyl)-ethyl]-methylamide hydrochloride.

In a further preferred aspect, the present invention provides a method for the treatment of depression and/or anxiety in a mammal including a human, which comprises treating said mammal with a therapeutically effective amount of a combination comprising a therapeutically non-effective dose of paroxetine hydrochloride anhydrate and a therapeutically non-effective dose of 2-(R)-(4-Fluoro-2-methyl-phenyl)(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide hydrochloride.

In a further preferred aspect, the present invention provides a method for the treatment of depression and/or anxiety in a mammal including a human, which comprises treating said mammal with a therapeutically effective amount of a combination comprising a therapeutically non-effective dose of paroxetine mesylate and a therapeutically non-effective dose of 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [-1-(R)(3,5-bis-trifluoromethyl-phenyl)-ethyl]-methylamide hydrochloride.

Reference herein to treatment extends to prophylaxis as well as to treatment of established depression and/or anxiety symptoms.

As used herein, the term depression includes depressive mood episodes, depressive disorders, bipolar disorders, other mood, psychotic, adjustment disorders premenstrual and dysphroic disorder(PMDD), Thus, for example, depressive mood episodes include major depressive episodes and mixed episodes. Depressive disorders include Major Depressive Disorder (MDD) single or recurrent episode (with or without psychotic features, catatonic features, melancholic features, atypical features, anxious depression, or postpartum onset), dysthymic disorder (with early or late onset and with or without atypical features) and depressive disorder not otherwise specified. Bipolar disorders include bipolar I and II disorders, cyclothymic disorder and bipolar disorder not otherwise specified. Other mood, psychotic and adjustment disorders include neurotic depression; mood disorders due to general medical conditions including, but not limited to, myocardial infarction, diabetes, miscarriage, abortion, premenstrual dysphroic disorders(PMDD), dementia of the Alzheimer's type (with early or late onset) with depressed mood, vascular dementia with depressed mood; substance-induced mood disorders including, but not limited to, depression induced by alcohol, amphetamines, cocaine, hallucinogens, inhalants, opioids, phencyclidines, sedatives, hypnotics, anxiolytics and other substances; schizoaffective disorder of the depressed type; adjustment disorder with depressed mood; adjustment disorder with mixed anxiety and depressed mood.

As used herein, the term anxiety includes panic attacks, agoraphobia, anxiety disorders, adjustment disorders and separation anxiety disorder and premenstrual dysphroic disorder(PMDD). Thus, for example, anxiety disorders include panic disorder with or without agoraphobia, agoraphobia without a history of panic disorder, specific phobia, social phobia (social anxiety disorder), obsessive-compulsive disorder, Acute and posttraumatic stress disorders, generalised anxiety disorders, anxiety disorder due to a general medical condition, substance-induced anxiety disorder, anxiety disorder not otherwise specified and mixed anxiety-depression disorders. Adjustment disorders include adjustment disorder with anxiety and adjustment disorder with mixed anxiety and depressed mood.

The advantageous profile of anti-anxiety activity obtained by the administration of a therapeutically non-effective dose of paroxetine or physiologically acceptable salts or solvates thereof with a therapeutically non-effective dose 2-(R)-(4-Fluoro-2-methyl-phenyl)-4-(S)-((8aS)-6oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or a pharmaceutically salt thereof can be demonstrated in the gerbil social interaction model, according to the method described by Cheeta et al. (Cheeta S. et al., 2001. Brain Research 915: 170-175).

It will be appreciated that the compounds of the combination may be administered simultaneously, either in the same or different pharmaceutical formulations, or sequentially. If there is sequential administration, the delay in administering the second and any subsequent active ingredient should not be such as to lose the benefit of a synergistic therapeutic effect of the combination of the active ingredients. It will also be understood that the compounds of the combination or the physiologically functional derivatives of any thereof, whether presented simultaneously or sequentially, may be administered individually or in multiples or in any combination thereof.

In a further preferred embodiment, the present invention provides the use of a therapeutically non-effective dose of paroxetine or physiologically acceptable salts or solvates thereof and a therapeutically non-effective dose of 2-(R)-(4-Fluoro-2-methyl-phenyl)-4-(S-((8aS)-6-oxo-hexahydro-pyrrolo[1,2-a]-pyrazin-2-yl)-piperidine-1-carboxylic acid [1-(R)-(3,5-bis-trifluoromethyl-phenyl)ethyl]-methylamide or pharmaceutically acceptable salt or solvates thereof in the manufacture of a therapeutically effective medicament for simultaneous or sequential administration for the treatment and/or prophylaxis of depression and/or anxiety.

In a further preferred embodiment, the present invention provides the use of a therapeutically non-effective dose of paroxetine, and a therapeutically non-effective dose of 2-(R)-(4-Fluoro-2-methyl-phenyl)-4-(S)((8aS) -6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide hydrochloride in the manufacture of a therapeutically effective medicament for simultaneous or sequential administration for the treatment and/or prophylaxis of depression and/or anxiety.

In a further preferred embodiment, the present invention provides the use of a therapeutically non-effective dose of paroxetine hydrochloride and a therapeutically non-effective dose of 2-(R)-(4-Fluoro-2-methyl-phenyl)-4-(S)-((8aS-6-oxo-hexahydro-pyrrolo[1,2-a]-pyrazin-2-yl)piperidine-1-carboxylic acid [-1-(R)-(3,5-bis-trifluoromethyl-phenyl)-ethyl]-methylamide hydrochloride in the manufacture of a therapeutically effective medicament for simultaneous or sequential administration for the treatment and/or prophylaxis of depression and/or anxiety.

In a further preferred embodiment, the present invention provides the use of a therapeutically non-effective dose of paroxetine hydrochloride hemihydrate and a therapeutically non-effective dose of 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide hydrochloride in the manufacture of a therapeutically effective medicament for simultaneous or sequential administration for the treatment and/or prophylaxis of depression and/or anxiety.

In a further preferred embodiment, the present invention provides the use of a therapeutically non-effective dose of paroxetine hydrochloride anhydrate and a therapeutically non-effective dose of 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide hydrochloride in the manufacture of a therapeutically effective medicament for simultaneous or sequential administration for the treatment and/or prophylaxis of depression and/or anxiety.

In a further preferred embodiment, the present invention provides the use of a therapeutically non-effective dose of paroxetine mesylate and a therapeutically non-effective dose of 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide hydrochloride in the manufacture of a therapeutically effective medicament for simultaneous or sequential administration for the treatment and/or prophylaxis of depression and/or anxiety.

The ratio of paroxetine or physiologically acceptable salts or solvates thereof to 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof, in the combination according to the invention may be for example, from 1:40 to 1:1 (measured by weight of the free bases).

The amount of a combination according to the invention required to be effective as an anti-depressive and/or anti-anxiety may, of course, vary and is ultimately at the discretion of the medical practitioner. The factors to be considered include the route of administration and nature of the formulation, the subject mammal's body weight, age and general condition and the nature and severity of the condition to be treated.

Unless otherwise indicated, all weights of active ingredients are calculated in terms of the drug per se. The desired dose may preferably be presented as one, two, three, four, five, six or more sub-doses administered at appropriate intervals throughout the day.

The components of the combination which may be referred to as active ingredients may be administered for therapy to an animal e.g. a mammal including a human in a conventional manner.

While it is possible for the active ingredients of the combination to be administered as the raw chemical, it is preferable to present them as a pharmaceutical formulation. Pharmaceutical formulations according to the present invention comprise a combination according to the invention together with one or more pharmaceutically acceptable carriers or excipients and optionally other therapeutic agents. The carrier(s) must be acceptable in the sense of being compatible with the other ingredients of the formula and not deleterious to the recipient thereof. When the individual components of the combination are administered separately, they are generally each presented as a pharmaceutical formulation. The references hereinafter to formulations refer, unless otherwise stated, to formulations containing either the combination or a component thereof.

A combination of paroxetine or physiologically acceptable salts or solvates thereof 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof, may conveniently be presented as a pharmaceutical formulation in a unitary dosage form.

Pharmaceutical formulations are often prescribed to the patient in “patient packs” containing the whole course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions and, therefore, lead generally to more successful treatment.

It will be understood that the administration of the combination of the invention by means of a single patient pack, or patient packs of each formulation, containing within a package insert instructing the patient to the correct use of the invention is a desirable additional feature of this invention.

According to a further aspect of the invention provided is a multiple, for example, double or triple, pack comprising at least paroxetine or physiologically acceptable salts or solvates thereof and 2-(R)-(4-Fluoro-2-methyl-phenyl)-4-(S-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide or pharmaceutically acceptable salts or solvates thereof, and an information insert containing directions on the use of the combination of the invention.

Formulations include those suitable for oral, rectal, nasal, topical (including transdermal, buccal and sublingual), vaginal or parenteral (including subcutaneous, intramuscular, intravenous and intradermal) administration. The formulations may conveniently be presented in unit dosage form and may be prepared by any methods well known in the art of pharmacy. Such methods represent a further feature of the present invention and include the step of bringing into association the active ingredients with the carrier which constitutes one or more accessory ingredients. In general, the formulations are prepared by uniformly and intimately bringing into association the active ingredients with liquid carriers or finely divided solid carriers or both, and then if necessary shaping the product.

Formulations of the present invention suitable for oral administration may be presented as discrete units such as capsules, caplets, cachets or tablets each containing a predetermined amount of the active ingredients; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredients in a free-flowing form such as a powder or granules, optionally mixed with a binder (e.g. povidone, gelatin, hydroxypropylmethyl cellulose), lubricant, inert diluent, preservative, disintegrant (e.g. sodium starch glycollate, sodium croscarmellose cross-linked povidone, cross-linked sodium carboxymethyl cellulose) surface-active or dispersing agent Molded tablets may be made by molding a mixture of the powdered compound moistened with an inert liquid diluent in a suitable machine. The tablets may optionally be coated or scored and may be formulated so as to provide slow or controlled release of the active ingredients therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredients in a flavored base, usually sucrose and acacia or tragacanth; pastilles comprising the active ingredients in an inert basis such as gelatin and glycerin, or sucrose and acacia; and mouthwashes comprising the active ingredients in a suitable liquid carrier. Formulations for rectal administration may be presented as a suppository with a suitable base comprising, for example, cocoa butter or polyethylene glycols. Topical administration may also be by means of a transdermal iontophoretic device.

Formulations suitable for vaginal administration may be presented as tablets, pessaries, tampons, creams, gels, pastes, foams or spray formulations containing in addition to the active ingredients such carriers as are known in the art to be appropriate.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of the active combination with the softened or melted carrier(s) followed by chilling and shaping in molds.

Formulations suitable for parenteral administration include aqueous and nonaqueous isotonic sterile injection solutions which may contain anti-oxidants, buffers, preservatives and solutes which render the formulation isotonic with the blood of the intended recipient; and aqueous and non-aqueous sterile suspensions which may include suspending agents and thickening agents; and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injection, immediately prior to use. Extemporaneous injection solutions and suspensions may be prepared from sterile powders, granules and tablets of the kind previously described.

It should be understood that in addition to the ingredients particularly mentioned above the formulations of this invention may include other agents conventional in the art having regard to the type of formulation in question, for example, those suitable for oral administration may include such further agents as sweeteners, thickeners and flavoring agents.

The pharmaceutical composition of the invention containing the two active ingredients may be prepared according to conventional techniques well known in the pharmaceutical industry. Thus, for example paroxetine or physiologically acceptable salts or solvates thereof and 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide or pharmaceutically acceptable salts or solvates thereof, may be admixed together with suitable excipients such as those described above for the formulation of each of the active ingredients separately. Tablets may be prepared, for example by direct compression of such a mixture or using other conventional methods. Bilayer tablets may be prepared according to conventional procedure. Thus, for example, by separately compressing the two blends in a suitable tabletting machine with two filling stations. Capsules may be prepared by filling the blend along with suitable excipients into gelatin capsules, using a suitable filling machine. Controlled release forms for oral or rectal administration may be formulated in a conventional manner associated with controlled release forms.

Paroxetine as the free base or in the form of any physiologically acceptable salt thereof, including all hydrated or anhydrous forms and all polymorphic forms of such salts may be prepared by the method described in U.S. Pat. No. 4,007,196, EP-B-0223403, EP-B-0808314 and EP-B-0970955 which are incorporated herein by reference hereto.

2-(R)-(4-Fluoro-2-methyl-phenyl)-4-(S)-((8aS)-6-oxo-hexahydro-pyrrolo[1,2-a]-pyrazin-2-ylpiperidine-1-carboxylic acid [1-(R)-(3,5-bis-trifluoromethyl-phenyl)ethyl]-methylamide or pharmaceutically acceptable salts or solvates thereof, may be prepared by the method described in WO 03/066635 which is incorporated herein by reference or by known methods described for analogous compounds.

For co-administration, paroxetine or physiologically acceptable salts or solvates thereof and 2-(R)-(4-Fluoro-2-methyl-phenyl)-4-(S-((8aS)-6-oxo-hexahydro-pyrrolo[1,2-a]-pyrazin-2-yl)-piperidine-1-carboxylic acid [-1-(R)-(3,5-bis-trifluoromethyl-phenyl)ethyl]-methylamide or pharmaceutically acceptable salts or solvates thereof, may be formulated in a conventional manner. Thus, for example paroxetine or physiologically acceptable salts or solvates thereof may be formulated as described in U.S. Pat. No. 4,007,196, EP-B0223403, EP-B-0808314 and EP-B-0970955 and 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof, may be formulated as described in WO 03/066635.

In a preferred aspect of the invention, paroxetine or physiologically acceptable salts or solvates thereof and 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)ethyl]-methylamide or pharmaceutically acceptable salts or solvates thereof, are formulated in a single pharmaceutical composition.

In order that this aspect of the invention may be more fully understood the following examples are given by way of illustration only.

In the following pharmaceutical formulation Compound A means 2-(R)-(4-Fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide or pharmaceutically acceptable salts or solvates thereof; Compound B means paroxetine or physiologically acceptable salts or solvates thereof

Tablets

Tablets may be prepared by the normal method such as direct compression or wet granulation.

The tablets may be film coated with a suitable film forming material such for example Opadry using standard technique.

### EXAMPLE 1

Direct Compression

Tablets

| | | |
|---|---|---|
| Compound A | 1 | mg (measured as the free base) |
| (as hydrochloride salt) | | |
| Compound B (as | 7.5 | mg(measured as the free base) |
| hydrochloride | | |
| hemihydrate salt) | | |
| Dibasic Calcium Phosphate | 106.75 | mg |
| Mg stearate | 1.5 | mg |
| Crospovidone | 4.5 | mg |
| Colloidal Silicon Dioxide: | 0.75 | mg |

Compound A, Compound B, dibasic Calcium Phosphate, Crospovidone, Colloidal Silicon Dioxide and Magnesium stearate are mixed together and the resultant mix is compressed into tablets using suitable machine so as to provide tablets according to example 1

### EXAMPLE 2

Wet granulation

| | | |
|---|---|---|
| Compound A | 1 | mg (measured as the free base) |
| (as hydrochloride salt) | | |
| Compound B | 7.5 | mg(measured as the free base) |
| (as hydrochloride | | |
| hemihydrate salt) | | |
| Microcrystalline cellulose | 103.75 | mg |
| Mg stearate | 1.5 | mg |
| Crospovidone | 4.5 | mg |
| Colloidal Silicon Dioxide | 0.75 | mg |
| Polyvinylpirrolidone | 3 | mg |

The compound A is mixed with Microcrystalline cellulose, Polyvinylpirrolidone and Crospovidone then granulated with a suitable amount of water. After drying the granule, the compound B and Colloidal Silicon Dioxide are added to it and mixed for a suitable time. The resulting mixture was blended with Mg stearate and then compressed into tablets as described in Example 1.

### EXAMPLE 3

Wet granulation

| | | |
|---|---|---|
| Compound A | 1 | mg (measured as the free base) |
| (as hydrochloride salt) | | |
| Compound B | 7.5 | mg(measured as the free base) |
| (as hydrochloride | | |
| hemihydrate salt) | | |
| Microcrystalline cellulose | 103.75 | mg |
| Mg stearate | 1.5 | mg |
| Crospovidone | 4.5 | mg |
| Colloidal Silicon Dioxide: | 0.75 | mg |
| Polyvinylpirrolidone | 3 | mg |

The compound B is mixed with Microcrystalline cellulose, Polyvinylpirrolidone and Crospovidone then granulated with a suitable amount of water. After drying the granule, the compound A and Colloidal Silicon Dioxide are added to it and mixed for a suitable time. The resulting mixture was blended with Mg stearate and then compressed into tablets as described in Example 1.

### EXAMPLE 4

co Wet granulation

| | | |
|---|---|---|
| Compound A | 1 | mg (measured as the free base) |
| (as hydrochloride salt) | | |
| Compound B | 7.5 | mg(measured as the free base) |
| (as hydrochloride | | |
| hemihydrate salt) | | |
| Microcrystalline cellulose | 103.75 | mg |
| Mg stearate | 1.5 | mg |
| Crospovidone | 4.5 | mg |
| Polyvinylpirrolidone | 3 | mg |

The compound B and the compound A are mixed with Microcrystalline cellulose, Polyvinylpirrolidone and Crospovidone then granulated with a suitable amount of water. After drying the granule, Mg stearate is added to it, blended and the resulting mixture compressed into tablets as described in Example 1

### EXAMPLE 5

Dry granulation

| | | |
|---|---|---|
| Compound A | 1 | mg (measured as the free base) |
| (as hydrochloride salt) | | |
| Compound B | 7.5 | mg(measured as the free base) |
| (as hydrochloride | | |
| hemihydrate salt) | | |
| Microcrystalline cellulose | 103.75 | mg |
| Mg stearate | 1.5 | mg |
| Crospovidone | 4.5 | mg |
| Colloidal Silicon Dioxide: | 1 | mg |
| Polyvinylpirrolidone | 2 | mg |

The compound B and the compound A are mixed with Microcrystalline cellulose, Mg stearate, Crospovidone, Colloidal Silicon Dioxide and Polyvinylpirrolidone. The resulting mixture is compressed with flat faced punches so as to provide slugs which fall into a mill so as to obtain granular particles. The granule is then compressed into tablets as described in example 1.

Pellets

### EXAMPLE 6

Extrusion-Spheronization

| | | |
|---|---|---|
| Compound A | 5 | mg (measured as the free base) |
| (as hydrochloride salt) | | |
| Compound B | 7.5 | mg(measured as the free base) |
| (as hydrochloride | | |
| hemihydrate salt) | | |
| Cellulose Spheres* | 109 | mg |
| Polyvinylpirrolidone | 4.5 | mg |

*Microcristalline cellulose (Avicel)

The compound B, after being mixed into the granulator chamber with microcrystalline cellulose, is wetted under agitation by spraying a suitable amount of water, the resulting wetted mass is extruded through a screen with proper dimensions so as to provide cylindrical extruded particles which are converted into pellets by the mechanical action of the rotating plate of a spheronizator. The pellets are dried and then encapsulated together with the compound A pellets produced applying the same process.

Alternatively, the compound B after being mixed into the granulator chamber with microcrystalline cellulose is wetted under agitation by spraying a suitable amount of water, the resulting mixture is agitated in order to let its particles growing up to pellets. The pellets are dried and then encapsulated together with the compound A pellets produced applying the same process.

Alternatively, a suitable amount of inert cellulose pellets are put in the fluid bed granulation chamber and set in motion introducing air at the bottom and then coated by spraying a water solution of the compound B. Pellets are dried and then encapsulated together with the compound A pellets produced applying the same process.

1-14. (canceled)

15. A method for the treatment of depression or anxiety in a human in need thereof comprising administering a therapeutically effective combination comprising a dose of each of components:
a) paroxetine or a physiologically acceptable salt or solvate thereof; and
b) 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide or a pharmaceutically acceptable salt or solvate thereof,
wherein said dose is lower than normally expected to produce an effective therapeutic response in the treatment of depression or anxiety in said human, as demonstrated in the gerbil social interaction model.

16. The method as claimed in claim 15 wherein said component a) is paroxetine hydrochloride hemihydrate salt and said component b) is 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [1-(R)-(3,5-bis-trifluoromethyl-phenyl)-ethyl]-methylamide hydrochloride salt.

17. The method as claimed in claim 15, wherein said dose of component a) is from 1 to 10 mg (measured as the free base).

18. The method as claimed in claim 15, wherein said dose of component a) is from 3.5 to 7.5 mg (measured as the free base).

19. The method as claimed in claim 15, wherein said dose of component b) is from 0.25 to 1 mg (measured as the free base).

20. The method as claimed in claim 15, wherein said dose of component b) is from 0.5 to 1 mg (measured as the free base).

21. The method as claimed in claim 15, wherein said dose of component a) is from 1 to 10 mg (measured as the free base) and said dose of component b) is from 0.25 to 1 mg (measured as the free base).

22. The method as claimed in claim 15, wherein said dose of component a) is from 1 to 10 mg (measured as the free base) and said dose of component b) is from 0.5 to 1 mg (measured as the free base).

23. The method as claimed in claim 15, wherein said dose of component a) is from 3.5 to 7.5 mg (measured as the free base) and said dose of component b) is from 0.25 to 1 mg (measured as the free base).

24. The method as claimed in claim 15, wherein said dose of component a) is from 3.5 to 7.5 mg (measured as the free base) and said dose of component b) is from 0.5 to 1 mg (measured as the free base).

25. The method as claimed in claim 15, wherein said combination is a unitary dosage form.

26. A pharmaceutical formulation comprising a dose of each of components:
a) paroxetine or a physiologically acceptable salt or solvate thereof; and
b) 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide or a pharmaceutically acceptable salt or solvate thereof,
wherein said dose of each component is lower than normally expected to produce an effective therapeutic response in the treatment of depression or anxiety in said human, as demonstrated in the gerbil social interaction model. together with one or more pharmaceutically acceptable carriers or excipients.

27. The pharmaceutical formulation as claimed in claim 26, wherein said component a) is paroxetine hydrochloride hemihydrate salt and said component b) is 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide hydrochloride salt.

28. The pharmaceutical formulation as claimed in claim 26 comprising:
a) from 3.5 to 7.5 mg (measured as the free base) of paroxetine hydrochloride hemihydrate salt and
b) from 0.5 to 1 mg (measured as the free base) of 2-(R)-(4-fluoro-2-methyl-phenyl)-4-(S)-((8aS)-6-oxo-hexahydro-pyrrolo[1,2-a]-pyrazin-2-yl)-piperidine-1-carboxylic acid [-1-(R)-(3,5-bis-trifluoromethyl-phenyl)-ethyl]-methylamide hydrochloride salt.
